(19) United States
(12) Patent Application Publication
(10) Pub. No.: US 2007/0141157 A1
(43) Pub. Date: Jun. 21, 2007

(54) Method for producing antibiotic/antibiotics and their use

(75) Inventors: Klaus-Dieter Kuhn, Marburg (DE); Sebastian Vogt, Erfurt (DE)

Correspondence Address: Kurt G. Briscoe;Norris, McLaughlin & Marcus P.A., 875 Third Avenue - 18th Floor, New York, NY, 10022, US

(73) Assignee: HERAEUS KULZER GMBH, Hanau (DE)

(21) Appl. No.: 11/441,656

(22) Filed: May 26, 2006

(30) Foreign Application Priority Data
Dec. 20, 2005 (DE) 10 2005 061 290.3

(51) Int. Cl. A61K 9/14

(52) U.S. Cl. 424/487; 424/489

(57) ABSTRACT

A method for producing antibiotic/antibiotics-particles characterized in that an aqueous solution of an amino glycoside-antibiotic or an aqueous solution comprising two or more amino glycoside-antibiotics is mixed under agitation with a solvent mixture comprising isopropanol and at least one additional alcohol, with the volume ratio of the solvent mixture to the aqueous solution being at least 3 to 1 and with the suspension developed being agitated until the primarily yielded coarse antibiotic/antibiotics aggregates disintegrate into particles, which have a grain size smaller than 400 μm.

The present invention relates to a method for producing antibiotics/antibiotics-particles and their use. Here, antibiotic/antibiotics-particles refer to particles with a grain size up to approximately 400 μm, in particular in the range from 63-400 μm.

Amino glycoside-antibiotics are typical, bactericidal broadband-antibiotics with a distinct post-antibiotic effect. Amino glycosides-antibiotics, in particular gentamicin in the form of gentamicin sulphate, are used for the antibiotic protection of polymethyl methacylate-bone cement (K.-D. Kuehn: Bone cements, Springer Verlag, 2000). Polymethyl methacrylate-bone cements serve in surgery and orthopedia for fixing endoprotheses. Here, there is the risk that after the hardening of the bone cement a germ infection can occur at the interface between the bone tissue and the polymethyl methacrylate-bone cement. An effective protection from a bacterial germ infection can be achieved by bactericidal antibiotic being inserted into the bone cement, which are dissolved from the hardened polymethyl methacrylate-bone cement by the influence of the aqueous physiological environment, and effectively kills sensitive bacterial germs at the interface between the bone tissue and the polymethyl methacrylate-bone cement.

Amino glycoside-antibiotics are industrially produced via biotechnology and/or sometimes synthetically with the use of micromonospora species, such as micromonospora purpurea. Here, amino glycoside-antibiotics are yielded in the form of a very fine powder with a typical grain size smaller than 64 μm, due to the normally used spray-drying methods. However, it has been shown that coarser particles are more advantageous for an optimum release of antibiotics from polymethyl methacrylate-bone cements. A grain size in the range from 63 to 250 μm is best.

Amino glycoside-antibiotics are generally used in the form of sulphates and easily dissolve in water. From U.S. Pat. No. 3,136,704 and U.S. Pat. No. 3,091,572, it is known to precipitate gentamicin sulphate from the aqueous solution using methanol. However, it has been shown in some experiments that no granular, easily isolated, coarse precipitation is yielded by mixing methanol with aqueous gentamincin sulphate-solutions. When mixing aqueous gentamicin sulphate-solutions with methanol or other lower alcohols, gentamicin sulphate precipitates in the form of an extremely sticky, slimy layer at the bottom of the reaction vessel. At the boundary surface between said layer and the solution positioned thereabove a skin forms, which hinders or prevents the dehydration of the gentamicin sulphate-slime. The layer solidifies no sooner than several days to weeks later. This layer of gentamicin sulphate can be further dehydrated and subsequently fractioned after being broken into the grain size desired. Using this process, the production of particular gentamicin sulphate is not economical. The sulphates and chlorides of the antibiotics tobramycin, amikacin, netilmicin, sisomycin, and kanamycin show a largely similar behavior as well.

The object of the invention is to provide a method by which it is possible to transfer in an economically advantageous, simple manner amino glycoside-antibiotics into a particular form with a grain size up to 400 μm, in particular in the range from 63 to 400 μm.

The object was attained such that an aqueous solution of an amino glycoside-antibiotic or an aqueous solution of two or more amino glycoside-antibiotics is mixed under agitation with a solvent mixture comprising isopropanol and at least one additional alcohol, with the volume ratio of the solvent mixture to the aqueous solution amounting to at least 3 to 1, and with the suspension yielded being agitated or being left standing until the primarily developing coarse antibiotic/antibiotics aggregates have disintegrated into particles with a grain size smaller than 400 μm.

The invention is based on the surprising result that mixtures made from 30-70 percent by volume isopropanol and 30-70 percent by volume methanol, when mixed with aqueous solutions of amino glycoside-antibiotics, first form coarse antibiotics aggregates of a size of approximately 1-10 mm, which disintegrate into particles with a grain size of <400 μm without any exterior influences, or accelerated by agitation, and with particles developing primarily having a grain size of 100-250 μm. The disintegration process runs both at room temperature as well as under elevated temperatures. A mixture of 50 percent by volume isopropanol and 50 percent by volume methanol has proven best. Using this mixture, the primarily formed antibiotics aggregates disintegrate within a range from a few minutes to a few hours. The antibiotic/antibiotics particles formed can be filtered off or centrifuged and, after additional dehydration steps, can be fractioned by sifting. This way, for example, the preferred sifting fraction is yielded having a grain size ranging from 63-400 μm. Within the scope of this method it is also possible to allow the disintegration of the primarily formed antibiotics aggregates at an elevated temperature.

Methanol is another preferred alcohol, in addition to isopropanol.

Therefore, a solvent mixture comprising 30-70 percent by volume isopropanol and 70-30 percent by volume methanol is preferred.

Particularly preferred is a solvent mixture comprising 50 percent by volume isopropanol and 50 percent by volume methanol.

The amino glycoside-antibiotic or the amino glycoside-antibiotics preferably originate from the group comprising gentamicin, tobramycin, amikacin, kanamycin, Netilmicin, and sisomycin.

The aqueous solution is preferably provided with an antibiotic/antibiotics-content ranging from 30 to 60 percent by mass. Solutions with a lower antibiotic/antibiotics-content are not economical and sometimes result in particles that are too fine. In higher contents of antibiotic/antibiotics, the aqueous solutions are highly viscous and hard to manipulate. In particular, the disintegration process of the primary aggregates lasts considerably longer than in the preferred range of concentration. The use of aqueous solutions with a content of antibiotic/antibiotics of 50 percent by mass has proven particularly advantageous.

In addition to one or more amino glycoside-antibiotics, the aqueous solution may also include one or more antibiotics of the groups of glycopeptide-antibiotics, the 4-quinolon antibiotics, the licosamide antibiotics, the macrolides, the ketolides, the glycylcyclines, and the linezolides or antimicrobially effective derivatives deducted therefrom. Advantageous antibiotics particles can be produced therefrom that are characterized in that a synchronized release of two or more antibiotics is possible after a radical hardening of the bone cements when they are used in polymethyl methacrylate-bone cements. This way, best levels of effective ingredients can be achieved, in particular of synergistically effective antibiotics.

The aqueous solution can additionally contain one or more polymers of the group of polyvinyl pyrolidon, vinyl alcohol, gelatin, carboxyl methyl cellulose, hydroxyl ethyl cellulose, methyl cellulose, and polyacryl acid salts in a dissolved form. The flow behavior of the antibiotics particles formed can be influenced by the added polymers.

Also included in the scope of the method according to the invention are still undissolved antibiotic/antibiotics-primary particles suspended in the aqueous antibiotic/antibiotics-solution. In the technical production of antibiotics particles it is possible to use antibiotic or antibiotics solutions that are not visually clear. Surprisingly, it has shown that still remaining remnants of primary particles do not negatively influence the formation of the desired coarse antibiotics particles.

The invention also relates to the use of antibiotics-particles produced according to the methods according to the invention for providing medical products and medicines with antibiotics, which are to be used for the localized release of an antibiotic or several antibiotics. Particularly preferred is the use of the antibiotics particles according to the invention for providing polymethyl methacrylate-bone cements with antibiotics.

The invention is explained in greater detail using the following examples, without limiting the invention thereby. Values of portions and percentages relate to weight, if not stated otherwise.

### BRIEF DESCRIPTION OF THE DRAWING

FIG. 1 shows a representation of the product according to example 1 using light-optical microscopy.

### EXAMPLES

### Example 1

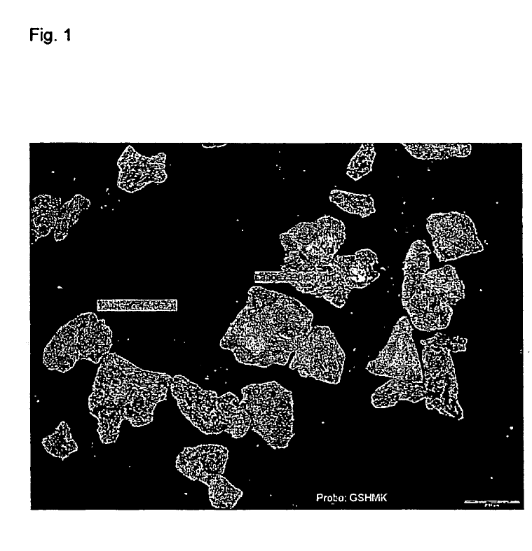

10.1 g gentamicin sulphate (AK 648, Fujiang/Fukang) is dissolved in 10.0 g distilled pyrogen-free water. This solution is added drop wise under agitation into a mixture of 50 ml isopropanol and 50 ml methanol. Within a few seconds, primary aggregates form. After half an hour it is no longer agitated. After four hours the sedimented primary aggregates have disintegrated. The particles formed are suctioned off, subsequently precipitated by agitation for half an hour in methanol at room temperature, and then dehydrated in a vacuum to the consistent weight. The overall yield amounts to 9.8 g. After sifting, 7.3 g particles are yielded with a sifting fraction from 63 to 250 μm. In FIG. 1 a representation of these particles is shown by light-optical microscopy.

### Example 2

10.0 g gentamicin sulphate (AK 648, Fujiang/Fukang) is dissolved in 10.0 g distilled pyrogen-free water. This solution is added drop wise under agitation into a boiling mixture of 50 ml isopropanol and 50 ml methanol under reflux. Within a few seconds primary aggregates form. After half an hour it is no longer agitated and the solvent mixture is left standing under reflux. The particles formed are cooled to room temperature after 5 hours, suctioned off, then precipitated by agitation within a half an hour in methanol at room temperature, and subsequently dehydrated in a vacuum to constant weight. The overall yield amounts to 9.6 g. After sifting, 6.6 g particles of the sifted fraction is yielded ranging from 63 to 250 μm.

### Example 3

7.7 g gentamicin sulphate (AK 648, Fujiang/Fukang) and 5.8 g clindamycin hydrochloride (AK 862) is dissolved in 10.0 g distilled pyrogen-free water. This solution is added drop wise into a mixture of 50 ml isopropanol and 50 ml methanol. Within a few seconds, primary aggregates form. After half an hour agitation is stopped. After five hours the sedimented primary aggregates have disintegrated. The particles formed are suctioned off, subsequently precipitated by agitation in methanol at room temperature for half an hour, and then dehydrated in a vacuum to constant weight. The overall yield amounts to 11.2 g. After sifting, 6.4 g particles of the sifting fraction ranging from 63 to 250 μm is yielded.

### Example 4

1.540 kg gentamicin sulphate (AK 648, Fujiang/Fukang) is dissolved in 1.0 kg distilled pyrogen-free water. In a 30 liter suspended vessel, 5.0 kg isopropanol and 5.0 kg methanol are provided. Into this solution the aqueous solution is added drop wise within thirty minutes under agitation (50 rotation per minute). During the drop wise addition, within a few seconds, primary aggregates form. After agitation at room temperature for four hours the primary aggregates are disintegrated. The particles formed are suctioned off, then precipitated by agitation for one hour in dry methanol at room temperature and subsequently dehydrated in a vacuum to constant weight. The overall yield amounts to 1.351 kg. After sifting, 0.899 kg particles is yielded of the sifted fraction ranging from 63 to 250 μm.

What is claimed is:

1. A method for producing antibiotic/antibiotics particles, comprising a) mixing an aqueous solution of an amino glycoside-antibiotic or an aqueous solution comprising two or more amino glycoside-antibiotics under agitation with a solvent mixture comprising isopropanol and at least one additional alcohol, with the volume ratio of the solvent mixture to the aqueous solution amounting to at least 3 to 1 to obtain a suspension comprising primarily coarse antibiotic/ antibiotics aggregates;
and b) agitating or leaving standing the suspension obtained until the coarse antibiotic/antibiotics aggregates disintegrate into particles having a grain size smaller than 400 μm.

2. A method according to claim 1, wherein the additional alcohol is methanol.

3. A method according to claim 1, wherein the amino glycoside antibiotic or the amino glycoside antibiotics are selected from the group consisting of gentamicin, tobramycin, amikacin, kanamycin, netilmicin, and sisomycin.

4. A method according to claim 1, wherein the solvent mixture comprises 30-70 percent by volume isopropanol and 70-30 percent by volume methanol.

5. A method according to claim 1, wherein the solvent mixture comprises 50 percent by volume isopropanol and 50 percent by volume methanol.

6. A method according to claim 1, wherein the aqueous solution has an antibiotic/antibiotics content ranging from 30-60 percent by weight.

7. A method according to claim 1, wherein the aqueous solution comprises, in addition to one or more amino glycoside antibiotics, one or more antibiotics selected from the group consisting of glycopeptide-antibiotics, 4-quinolon antibiotics, lincosamide antibiotics, makrolides, ketolides, glycylcyclines, and linezolides and antimicrobially effective derivatives thereof.

8. A method according to claim 1, wherein the aqueous solution additionally comprises one or more polymers selected from the group consisting of polyvinyl pyrolidon, vinyl alcohol, gelatin, carboxyl methyl cellulose, hydroxyl ethyl cellulose, methyl cellulose, and polyacryl acid salts in a dissolved form.

9. A method according to claim 1, wherein still undissolved antibiotic/antibiotics primary particles are suspended in the aqueous antibiotic/antibiotics solution.

10. Antibiotic/antibiotics particles produced by the method according to claim 1, having a grain size distribution in the range of 63-400 μm.

11. A method of treating a bacterial infection in a patient in need thereof comprising administering to said patient antibiotic/antibiotics particles according to claim 10.

12. A polymethyl methacrylate-bone cement comprising an antibacterially effective amount of antibiotic/antibiotics particles according to claim 10.
